(19) United States
(12) Patent Application Publication
(10) Pub. No.: US 2026/0182919 A1
(43) Pub. Date: Jul. 2, 2026

(54) PRESSURE SENSING GUIDEWIRE DEVICE AND METHODS OF USE THEREOF

(71) Applicant: Icahn School of Medicine at Mount Sinai, New York, NY (US)

(72) Inventors: Alexis Angelo Jacques Bruhat, New York, NY (US); Daniel Ginberg, New York, NY (US); Pierre Jean Cottinet, New York, NY (US)

(73) Assignee: Icahn School of Medicine at Mount Sinai, New York, NY (US)

(21) Appl. No.: 18/868,047

(22) PCT Filed: May 25, 2023
(86) PCT No.: PCT/US2023/067449

§ 371 Date: Nov. 21, 2024

Related U.S. Application Data
(60) Provisional application No. 63/347,239, filed on May 31, 2022.

(51) Int. Cl. A61B 5/00; A61B 5/02; A61M 25/00; A61M 25/09

(52) U.S. Cl. CPC A61B 5/6851; A61B 5/02028; A61B 5/7214; A61B 2505/05; A61B 2562/0247; A61B 2562/043; A61M 2025/0002; A61M 2025/09166; A61M 2025/09175

(57) ABSTRACT

Disclosed herein are pressure-sensing guidewire devices, systems, and methods that enable measurement of coaptation forces between tissues.

### CROSS-REFERENCE TO RELATED APPLICATION

This application claims the benefit of U.S. Provisional Application No. 63/347,239 filed May 31, 2022, the disclosure of which is hereby incorporated by reference in its entirety.

### FIELD OF THE INVENTION

The present invention relates generally to measuring, quantifying, and displaying the coaptation or closure force between at least two tissues in a patient.

### BACKGROUND

Mitral regurgitation (MR) is a failure of the sealing function of mitral valve (MV) leaflets. MR occurs in roughly 2% of the US population and its incidence increases with age. Primary lesions are the most frequent mechanism of MR. In this etiology, MV repair (MVR) under cardiac arrest is the gold-standard treatment. The goal of MVR is to restore a proper contact between the two mitral leaflets (so called coaptation phenomena) in order to ensure the sealing function and to decrease global valvular stress. In transcatheter MVR (T-MVR), more than 20% of patients will have regurgitation in the next months after operation.

Currently, there are limited methods for intraoperative (e.g. on an empty and stopped heart) assessment of the quality of the MVR. One current technique includes inflating the left ventricle with saline liquid (a “saline test”) to mimic ventricular systole resulting in the closing of the mitral leaflets. The saline test is also used to subjectively and globally assess coaptation function. Another test includes “painting” the appearing surface of mitral leaflets (atrial surface) during the saline test. Thus, when the ventricular content is subsequently sucked, the coaptation surface is the only valvular part appearing without ink. This “ink test” provides an approximate assessment of the height of coaptation. However, both of these tests are subjective and approximate and therefore rely on the expertise of the surgeon. Therefore, MVR success rates and outcomes mainly rely on surgical expertise and the consequences of MVR failure strongly impact survival.

Currently, the only definitive assessment of MVR is obtained after closure of the heart cavities and after withdrawal of the cardiopulmonary bypass. An intraoperative transesophageal echocardiography is systematically performed to assess the MVR results (e.g., the quality of the valvular sealing function and morphological description of leaflet morphology). Echocardiography allows a two-dimensional morphological assessment of the coaptation in the cut plane of ultrasound but it is unable to show the global render of the coaptation surface, as well as the coaptation forces. In the case of obvious immediate failure of MVR, additional repair techniques could be performed but sometimes the valve needs to be replaced. Additionally, even with a good intraoperative echocardiographic result, early failure of MVR can happen.

Fractional flow reserve (FFR) devices are commonly used to measure blood pressure and temperature from within the heart cavities. Current FFR devices embed enclosed piezoresistive sensors at the tip of guidewires to reduce signal noise from contact on the vessel walls, making their design purposely unable to perform mitral valve coaptation forces (MCF) measurements.

Accordingly, there is a need for a device allowing real-time, intraoperative MCF measurements to increase repair rate and repair success rate.

### BRIEF SUMMARY

The disclosure provides for systems and methods for measuring a coaptation force of two tissues in a patient. The two tissues may be valvular tissues within the patient's heart, such as but not limited to leaflets of a mitral valve, an aortic valve, a pulmonary valve, or tricuspid valve.

In some aspects, the device may include a guidewire body comprising at least two cavities at a distal end of the guidewire body; at least two pressure sensors, each pressure sensor configured to fit within a corresponding cavity on the guidewire body; and a flexible coating covering the at least two pressure sensors.

The at least two pressure sensors are configured to provide a measurement of the coaptation force of the two tissues.

In various aspects, the flexible coating fills a gap in each cavity between the top of each pressure sensor and a surface of the guidewire body. The flexible coating may be flush with the surface of the guidewire body. The flexible coating may extend above the surface of the guidewire body. In an aspect, the device may further include a second coating configured to cover the flexible coating over the at least two pressure sensors and the surface of the guidewire body. Each cavity has a depth of 0.10 mm to 0.13 mm. Each pressure sensor has a height of 0.05 mm to 0.08 mm. The gap between the top of each pressure sensor and the surface of the guidewire body has a depth of 0.02 mm to 0.08 mm.

In some aspects, the device may include three or four pressure sensors. At least one of the at least two pressure sensors may be located along a sensing area at the distal end of the guidewire body, and at least one pressure sensor in the sensing area may be configured to measure mechanical pressure between the two tissues. At least one of the at least two pressure sensors may be located outside of the sensing area at the distal end of the guidewire body, and the pressure sensor outside the sensing area may be configured to measure blood pressure outside the sensing area. A measurement from the at least one pressure sensor outside the sensing area may be used to denoise a measurement from the at least one pressure sensor in the sensing area to generate the measurement of the coaptation force of the two tissues. The sensing area may be 1 cm to 1.5 cm long. The at least one pressure sensor outside the sensing area may be proximal to the sensing area along the guidewire body. The at least one pressure sensor outside the sensing area may be 1 cm to 2 cm from the sensing area. The device may further include a radiopaque tip distal to the sensing area and/or a radiopaque marker proximal to the sensing area.

In some aspects, the at least two pressure sensors may be piezoresistive force sensors. The at least two cavities may each comprise a sensor housing. The device may further include a handle configured to hold a processor, a battery, and a wireless transmitter.

Further provided herein is a method of measuring a coaptation force of two tissues in a patient. The method may include positioning a pressure-sensing guidewire device between the two tissues and measuring the coaptation force of the two tissues via the at least two pressure sensors. In some examples, the two tissues may be valvular tissues within the patient's heart and the pressure-sensing guidewire device measures pressures in a beating heart. The valvular tissues may include but are not limited to leaflets of a mitral valve, an aortic valve, a pulmonary valve, or tricuspid valve. In other examples, the two tissues comprise a sphincter, a stenosis of a blood vessel, or a vascular closure.

In some aspects, the method may further include inserting the pressure-sensing guidewire device endovascularly and/or monitoring the position of the pressure-sensing guidewire device and the at least two pressure sensors. Monitoring the position of the device may include using fluoroscopy, ultrasound, a pressure waveform from the at least two pressure sensors, or a combination thereof.

In various aspects, the method may further include positioning a first pressure sensor between the two tissues, where a second pressure sensor is not between the two tissues, receiving a first pressure measurement from the first pressure sensor and a second pressure measurement from the second pressure sensor, denoising the first pressure measurement, using the second pressure measurement, to generate the measurement of the coaptation force of the two tissues, generating a visual representation of the coaptation force between the two tissues from the measured pressures, and/or comparing elements of the visual representation to determine the efficacy of the coaptation.

Additional aspects and features are set forth in part in the description that follows, and will become apparent to those skilled in the art upon examination of the specification or may be learned by the practice of the disclosed subject matter. A further understanding of the nature and advantages of the disclosure may be realized by reference to the remaining portions of the specification and the drawings, which forms a part of this disclosure.

### DESCRIPTION OF THE DRAWINGS

The description will be more fully understood with reference to the following figures, which are presented as variations of the disclosure and should not be construed as a complete recitation of the scope of the disclosure, wherein:

FIG. 1 shows the pressure-sensing guidewire device in one example.

FIG. 2 shows the pressure-sensing guidewire device in one example.

FIG. 3 shows the electrical architecture of the pressure-sensing guidewire device in one example.

FIG. 4 shows an example computing system.

FIG. 5 shows a sample 2D map from the pressure-sensing guidewire device in one example.

FIG. 6 shows data from a commercial FFR device used off-label to record clinical MCF.

FIG. 7A and FIG. 7B show sample measurements from a prototype pressure-sensing guidewire device.

### DETAILED DESCRIPTION

The pressure-sensing guidewire device and method of use will be understood, both as to its structure and operation, from the accompanying drawings, taken in conjunction with the accompanying description. It is noted that, for purposes of illustrative clarity, certain elements in various drawings may not be drawn to scale. Several variations of the device are presented herein. It should be understood that various components, parts, and features of the different variations may be combined together and/or interchanged with one another, all of which are within the scope of the present application, even though not all variations and particular variations are shown in the drawings. It should also be understood that the mixing and matching of features, elements, and/or functions between various variations is expressly contemplated herein so that one of ordinary skill in the art would appreciate from this disclosure that the features, elements, and/or functions of one variation may be incorporated into another variation as appropriate, unless described otherwise.

Several definitions that apply throughout this disclosure will now be presented. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

The terms “comprising” or “having” mean “including, but not necessarily limited to”; specifically indicate open-ended inclusion or membership in a so-described combination, group, series and the like. The terms “comprising” and “including” as used herein are inclusive and/or open-ended and do not exclude additional, unrecited elements or method processes. The term “consisting essentially of” is more limiting than “comprising” but not as restrictive as “consisting of.”Specifically, the term “consisting essentially of” limits membership to the specified materials or steps and those that do not materially affect the essential characteristics of the claimed invention. The terms “a,” “an,” and “the” are understood to encompass the plural as well as the singular.

For purposes of this description, “distal” refers to the end extending into a body and “proximal” refers to the end extending out of the body.

For purposes of this description, “connected to” includes two components being directly connected or indirectly connected with intervening components.

For purposes of this description, “coaptation” includes the closing or drawing together of separated tissue. Coaptation may occur in a wound, fracture, or in a functioning tissue such as valves in the heart. In a physiologic state during the systole the two mitral leaflets encounter each other in a meeting zone (the coaptation phenomena) ensuring proper sealing. Proper coaptation prevents blood regurgitation from left ventricle to the left atrium. In tri-leaflet valves (aortic and tricuspid valve), the sealing of the valve is ensured by the proper configuration of all leaflets. However, in the tri-leaflet valves, the coaptation still occurs between two leaflets aside from the very center of the valve where the coaptation may occur between the three leaflets.

A “coaptation force” includes the pressure or closure force between the two tissues when the tissues are closed together. For example, the coaptation force may be measured along the contact surface between the two tissues.

As used herein, a “measurement” from a pressure sensor includes a signal or a waveform. “Measurement”, “plurality of measurements”, “pressure signal”, and “pressure waveform” may be used synonymously.

The terms used in this specification generally have their ordinary meanings in the art, within the context of the disclosure, and in the specific context where each term is used. Alternative language and synonyms may be used for any one or more of the terms discussed herein, and no special significance should be placed upon whether or not a term is elaborated or discussed herein. In some cases, synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any example term. Likewise, the disclosure is not limited to various embodiments given in this specification.

Disclosed herein is a pressure-sensing guidewire device and system and methods of use thereof that enables the measurement of coaptation or closure forces between at least two tissues. The pressure-sensing guidewire device is a minimally invasive (endovascular) diagnostic device operable to enable intraoperative measurement of mitral valve coaptation forces (MCF) during mitral valve repair (MVR). MCF has been shown to be an indicator of mitral valve function and may be a prognostic indicator of long term repair stability. No current device is capable of accurately measuring MCF intraoperatively.

Surgical trends and outcome data increasingly favor minimally invasive (endovascular) approaches, as opposed to traditional “open” surgery (O-MVR). With growing support for transcatheter MVR (T-MVR) technologies, there will likely be a growing caseload of endovascular MVR. Therefore, there is a need for an MCF sensor device to meet both T-MVR and O-MVR needs. The pressure-sensing guidewire device described herein is designed to measure and record MCF during T-MVR and O-MVR and without disrupting the procedure. This device is fundamentally different from other intracardiac pressure sensor devices by ensuring accurate and comprehensive MCF data collection through the integration of at least two pressure sensors, innovative coating designs, application-specific guidewires, advanced micro-interconnect communication systems, and a surgeon-friendly data analysis interface.

In an example, the pressure-sensing guidewire system may include a pressure-sensing guidewire device. In some examples, the pressure-sensing guidewire system further includes a computing system. In addition, the pressure-sensing guidewire device enables measurement of the coaptation force in vivo and allows real-time assessment of the success of surgical repair to at least one of the tissues.

The pressure-sensing guidewire device is used to objectively measure the coaptation force between two tissues in a patient's body. For example, the two tissues may be within the patient's heart. Non-limiting examples of tissues include valvular tissues such as leaflets of a mitral valve, an aortic valve, a pulmonary valve, or a tricuspid valve. The pressure-sensing guidewire device may be used intraoperatively to provide an objective measurement of the coaptation force between the two tissues after repair to at least one tissue. For example, prior methods of assessing mitral valve repair were subjective or only provided high level information regarding inadequate coaptation. These assessment levels are not quantitative and do not provide localized information as to where a correction may need to occur. The objective measurement of the coaptation force provides detailed intraoperative information to the surgeon to determine whether the repair was sufficient, whether further adjustments are needed prior to completion of the surgery, and where the repair insufficiencies are located.

In some embodiments, the pressure-sensing guidewire device is deployable using standard vascular catheter techniques, identifiable and maneuverable under fluoroscopic and echocardiographic guidance, fits alongside T-MVR and O-MVR repair devices, collects and transmits real-time precise MCF measurements, and does not interfere with mitral leaflet functionality.

### I. Pressure-Sensing Guidewire Device

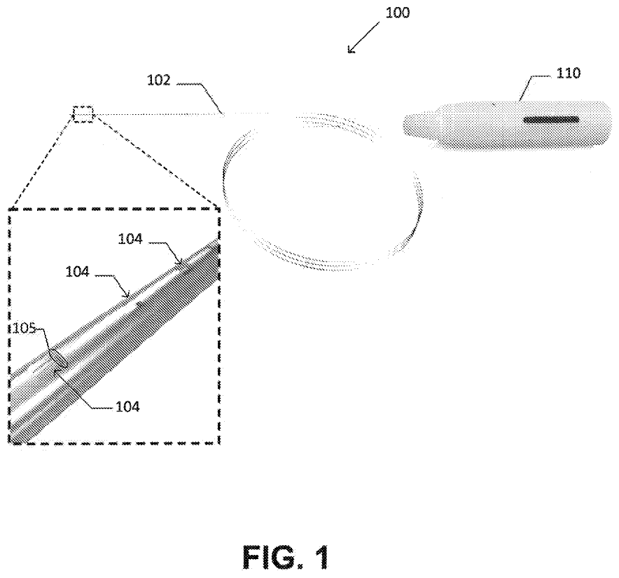

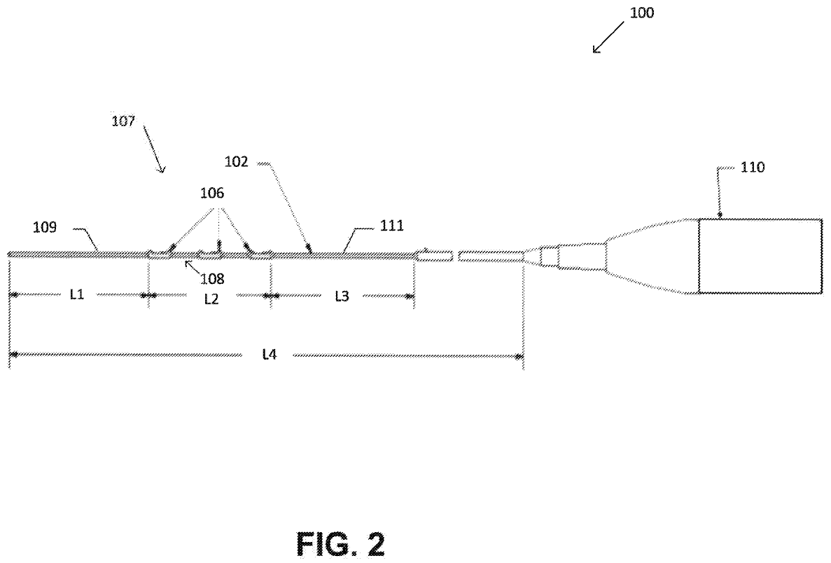

FIGS. 1 and 2 depict a pressure-sensing guidewire device 100 in one embodiment. In some embodiments, a pressure-sensing guidewire system may include a pressure-sensing guidewire device 100 and a computing system 200. It will be appreciated that the pressure-sensing guidewire system and/or pressure-sensing guidewire device 100 overcomes one or more of the above-listed problems commonly associated with conventional means for observing and evaluating valve closure.

As seen in FIGS. 1 and 2, the pressure-sensing guidewire device 100 includes a guidewire body 102 and at least two pressures sensors 104 that are configured to provide a measurement of the coaptation force of two tissues in the body of a patient. In some examples, the pressure-sensing guidewire device 100 may be inserted into the heart of the patient. In various examples, the pressure-sensing guidewire device 100 may be inserted into a heart that is under cardiopulmonary bypass. In another embodiment, the pressure-sensing guidewire device 100 may be inserted into the heart through percutaneous surgery, without cardiopulmonary bypass. In yet another embodiment, the pressure-sensing guidewire device 100 may be endovascularly inserted into the heart during transcatheter surgery. In some examples, the heart is currently undergoing or has recently undergone surgery to repair at least one tissue in the heart. For example, the pressure-sensing guidewire device 100 may be placed between atrioventricular valves in the heart during surgery, post-surgical repair of at least one of the atrioventricular valves, or after closure of the atrium. In this example, the pressure-sensing guidewire device 100 is placed between the leaflets of the mitral valve. The pressure-sensing guidewire device 100 is designed to minimize interference with mitral coaptation so as to not induce temporary regurgitation during deployment. The various aspects of the pressure-sensing guidewire device 100 are waterproof and biocompatible for use in surgery.

As seen in FIGS. 1 and 2, the pressure-sensing guidewire device 100 may include a guidewire body 102 comprising at least two cavities 106 along a sensing area 108 at a distal end of the guidewire body 102, at least two pressure sensors 104, and a flexible coating covering the at least two pressure sensors 104.

The distal end 107 of the guidewire body 102 may include a radiopaque distal tip 109, a sensing area 108, and/or an intermediate portion 111. The distal end 107 may have a length of the total length of the radiopaque distal tip L1, length of the sensing area L2, and length of the intermediate portion L3. For example, the distal end 107 of the guidewire body 102 may have a length ranging from about 3 cm to about 8 cm, about 3 cm to 5 cm, about 4 cm to 6 cm, about 5 cm to 7 cm, or about 6 cm to 8 cm. For example, the distal end 107 may have a length of about 3 cm, about 4 cm, about 5 cm, about 6 cm, about 7 cm, or about 8 cm. In at least one example, the distal end 107 may have a length of about 6.2 cm.

The at least two cavities 106 in the guidewire body 102 allows for inset of the pressure sensors 104. The cavities 106 may be located in the sensing area 108 and/or the intermediate portion 111. Each pressure sensor 104 may be configured to fit within a corresponding cavity 106 on the guidewire body 102. In some embodiments, each cavity has a depth of about 0.10 mm to 0.13 mm. In some embodiments, the cavity 106 is within a pressure sensor housing. The pressure sensor housing may have an outer diameter wider than the outer diameter of the guidewire body 102. For example, the pressure sensor housing may have an outer diameter of about 0.01 in to about 0.015 in. In at least one example, the pressure sensor housing may have an outer diameter of about 0.014 in.

Non-limiting examples of pressure sensors 104 include resistive, piezoresistive, capacitive, electromagnetic, piezoelectric, optical, or potentiometric pressure/force sensors. The pressure sensors may be configured to detect a minimum pressure of 2 gF. In an embodiment, the at least two pressure sensors 104 measure forces, such as pressure of at least about 2 gF. In an embodiment, the at least two pressure sensors 104 measure pressures of less than about 5 gF. In an embodiment, the at least two pressure sensors 104 measure pressures of less than about 10 gF.

In some embodiments, the pressure sensors may include advanced piezoresistive gauge technology to allow for ultra-precise measurements of MCF. Strain gauges may include specific properties in terms of linearity, hysteresis, and sensitivity allowing for their use within the mitral valve. Semiconductor strain gauges may be optimized in terms of geometry, and material properties may minimize the limitations of device drift.

In various embodiments, the at least 2 pressure sensors 104 may include 2 pressure sensors, 3 pressure sensors, 4 pressure sensors, 5 pressure sensors, 6 pressure sensors, 7 pressure sensors, or at least 8 pressure sensors. In one embodiment, as seen in FIGS. 1 and 2, the at least two pressure sensors may include 3 pressure sensors 104 arranged in a row in the sensing area 108 and/or intermediate portion 111 at a distal end 107 of the guidewire body 102. In an embodiment, the pressure sensors 104 are strategically placed on the guidewire body 102 near the distal end to allow for synchronistic recording of the left atrial, the mitral coaptation, and the left ventricular signal.

The at least two pressure sensors 104 are configured for measuring pressures both inside and outside the coaptation. In addition to the mechanical forces between the two tissues, there may be other pressures, such as blood pressure, around the tissues that could influence the measurement of the mechanical force between the two tissues. A pressure sensor outside the coaptation of the two tissues may be used as a baseline for measuring the external pressures, such as blood pressure. The baseline measurement can then be used to denoise the measurement from the pressure sensor(s) between the two tissues to give a more accurate reading of mechanical force of the coaptation. For example, when the device is placed such that at least one pressure sensor 104 is between the two tissues and at least one pressure sensor 104 is not between the two tissues, the pressure sensor 104 outside the tissues can be used to denoise the measurement from the pressure sensor(s) 104 between the tissues. This acquisition performs real-time signal denoising thus ensuring an adequate device placement, a precise MCF measurement, and an estimation of the coaptation height. In some examples, a first pressure measurement from one or more pressure sensors 104 in the sensing area 108 may be denoised using a second pressure measurement from one or more pressure sensors 104 in the intermediate portion 111 by subtraction, advanced filtering, and/or functional dependencies between the first and second sensor waveforms.

In some embodiments, at least one of the at least two pressure sensors 104 is located along a sensing area 108 at the distal end 107 of the guidewire body 102 and at least one of the at least two pressure sensors 104 is located outside of the sensing area 108 (e.g. located in the intermediate portion 111). The pressure sensor(s) 104 in the sensing area 108 are configured to measure mechanical pressure between the two tissues. The pressure sensor(s) 104 located in the intermediate portion 111 are configured to measure blood pressure outside the sensing area 108.

At least one of the at least two pressure sensors 104 may be located along a sensing area 108 at the distal end 107 of the guidewire body 102. In some embodiments, 1, 2, 3, 4, or 5 pressure sensors 104 may be located within the sensing area 108. The sensing area 108 may be configured to be placed between the two tissues and measure the coaptation between the two tissues. The sensing area 108 may have a length L2 of about 0.5 cm to about to about 5 cm, 0.5 cm to 1 cm, 1 cm to 1.5 cm, 1.5 cm to 2 cm, 2 cm to 2.5 cm, 2.5 cm to 3 cm, 3 cm to 3.5 cm, 3.5 cm to 4 cm, 4 cm to 4.5 cm, or 4.5 cm to 5 cm. In at least one example, the sensing area 108 may have a length L2 of 1.2 cm. In some embodiments, the pressure sensors 104 may be spaced evenly within the sensing area 108.

The intermediate portion 111 may transition the distal end 107 to the rest of the guidewire body 102. In an embodiment, one or more pressure sensors 104 may be located on the intermediate portion 111 to measure a baseline of external pressures, such as blood pressure outside the coaptation. In some embodiments, 1, 2, 3, 4, or 5 pressure sensors 104 may be located within the intermediate portion 111. The intermediate portion 111 may be configured to not be located between the two tissues when the sensing area 108 is located between the two tissues. The measurement from a pressure sensor 104 in the intermediate portion 111 may be used to denoise the measurement from the pressure sensor(s) 104 in the sensing area 108. In some embodiments, the intermediate portion 111 may extend a length L3 along the distal end 107 of the guidewire body 102. The intermediate portion 111 may have a length L3 of about 0.5 cm to about 5 cm, 0.5 cm to 1 cm, 1 cm to 1.5 cm, 1.5 cm to 2 cm, 2 cm to 2.5 cm, 2.5 cm to 3 cm, 3 cm to 3.5 cm, 3.5 cm to 4 cm, 4 cm to 4.5 cm, or 4.5 cm to 5 cm. In at least one embodiment, the intermediate portion 111 has a length of 2 cm.

In an embodiment, each pressure sensor may have a length of at least about 0.5 mm. In an embodiment, each pressure sensor may have a length of at least about 1 mm. In an embodiment, each pressure sensor may have a length of at least about 2 mm. In an embodiment, each pressure sensor may have a length of at least about 4 mm. In an embodiment, each pressure sensor may have a length of at least about 5 mm. In an embodiment, each pressure sensor may have a length of less than about 5 mm.

Each pressure sensor may have a height of about 0.05 mm to 0.08 mm. There may be a gap between the top of each pressure sensor 104 and the surface of the guidewire body 102. In some embodiments, the gap may have a depth of about 0.02 mm to 0.08 mm.

The pressure-sensing guidewire device 100 further includes a flexible coating covering the at least two pressure sensors 104 to allow for accurate transmission of mechanical stresses. The flexible coating fills a gap in each cavity 106 between the top of each pressure sensor 104 and a surface of the guidewire body 102. In an embodiment, the flexible coating is flush with the surface of the guidewire body 102. In another embodiment, the flexible coating extends above the surface of the guidewire body 102. The flexible coating may include a material that is biocompatible, hydrophobic, and compatible with sterilization. Non-limiting examples of flexible coating materials include parylene C (with a high young modulus) and polydimethylsiloxane (PDMS) (with a low young modulus).

Unlike the case of FFR sensors, where the device must measure blood pressure without contact with vascular or heart tissue, the goal of pressure-sensing guidewire devise is the opposite: measuring the forces of contact between two heart tissues. Therefore, a dedicated interface surrounding the sensor (e.g. the flexible coating) is needed to allow for accurate transmission of mechanical stresses between the mitral valve and the sensor. The flexible coating allows for pressures on the coating to be transferred to the pressure sensors for accurate measurement of pressures. Without the flexible coating, the inset pressure sensors 104 in the cavities 106 of the guidewire body 102 would not be able to contact the tissues or accurately measure pressures, such as the MCF.

The flexible coating may have half-sphere type shape over the pressure sensors to maximize transmission of mechanical stress without influencing the shape of the mitral valve. The flexible coating may be biocompatible and have a Young's modulus range of between 1 MPa to 100 MPa such that it can sufficiently transfer force without affecting pressure measurements at the pressure sensors.

In some embodiments, the pressure-sensing guidewire device 100 further includes a second coating configured to cover the flexible coating over the at least two pressure sensors 104 and the surface of the guidewire body 102. The second coating provides a continuous surface over the entire guidewire body 102 that will be inserted into the patient. It also provides an additional barrier to waterproof the device. The second coating may include a material that is biocompatible, hydrophobic, and compatible with sterilization. Non-limiting examples of second coating materials include butadiene rubber (NBR) and polydopamine (PDA).

The pressure-sensing guidewire device 100 may further include advanced micro-interconnects 105 and low-wire count communication to enable successful data transfer of the pressure sensors 104. In some embodiments, the micro-interconnects 105 may be located within each cavity 106 with each pressure sensor 104. The integration of the sensor and the interface layer is especially complex in a guidewire with radio and acoustic detection elements. Compact application-specific electronics at the tip that drive the devices and enable multiplexing and processing locally to avoid losses and interference over the long distances of the catheter (˜1 m).

The pressure-sensing guidewire device 100 is also configured for navigation under fluoroscopic guidance into the left atrium (LA) and mitral valve with the assistance of a radiopaque tip distal to the sensing area. The radiopaque distal tip 109 may have a length L1 of about 0.5 cm to about 5 cm, 0.5 cm to 1 cm, 1 cm to 1.5 cm, 1.5 cm to 2 cm, 2 cm to 2.5 cm, 2.5 cm to 3 cm, 3 cm to 3.5 cm, 3.5 cm to 4 cm, 4 cm to 4.5 cm, or 4.5 cm to 5 cm. In at least one embodiment, the radiopaque distal tip has a length of 2 cm. In an embodiment, the guidewire body 102 may further include a radiopaque marker proximal to the sensing area 108. For example, the guidewire body 102 may have a radiopaque marker proximal as well as distal (e.g. radiopaque distal tip 109) to the valve (i.e. sensing area 108), so that it can be confirmed that the pressure sensors 104 are being contacted by the valve leaflet (between the radiopaque markers). In some embodiments, the position of the device and the pressure sensors may be monitored using fluoroscopy, ultrasound, a pressure waveform from the at least two pressure sensors, or a combination thereof.

The guidewire body 102 may have a length of about 90 cm to about 150 cm. In various embodiments, the guidewire body 102 may have a length of 90 cm to 110 cm, 100 cm to 120 cm, 110 cm to 130 cm, 120 cm to 140 cm, 130 cm to 140 cm, or 130 cm to 150 cm. In at least one example, the guidewire body 102 may have a length of 120 cm.

The gauge of the guidewire body 102 may allow the pressure-sensing guidewire device to be stiff enough to be pushed through a catheter or valve. The guidewire body 102 may have an outer diameter ranging from 0.005 in to 0.040 in. In various embodiments, the guidewire body 102 may have an outer diameter of 0.008 in to 0.012 in, 0.010 in to 0.020 in, 0.015 in to 0.025 in, 0.020 in to 0.030 in, 0.025 in to 0.035 in, or 0.030 in to 0.040 in. In at least one example, the guidewire body 102 may have an outer diameter of 0.010 in.

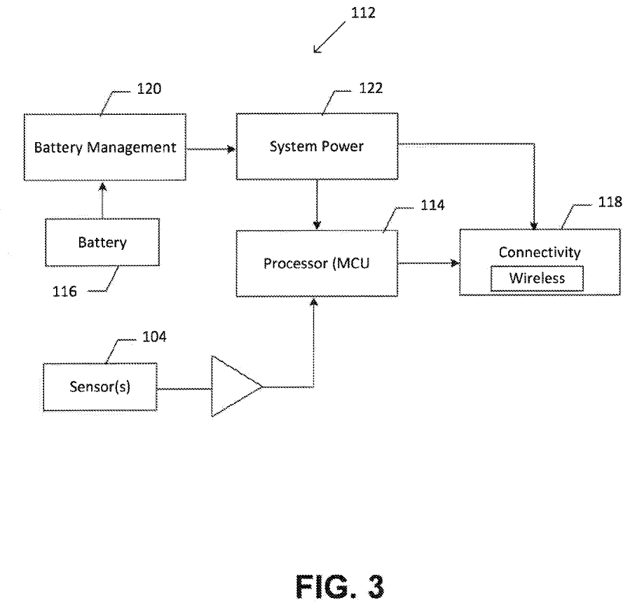

The pressure-sensing guidewire device 100 may further include a housing 110 configured to hold a processor (e.g. MCU) 114, a battery 116, and a wireless transmitter (wireless communicator) 118. The processor 114 may be configured for receiving the measurements received by the at least two pressure sensors 104 and the wireless transmitter may be configured for outputting the received measurements. FIG. 3 shows the electrical architecture 112 within the housing of the pressure-sensing guidewire device 100. The processor 114 may be powered by the battery 116, via the battery management module 120 and system power module 122. The battery 116 may be an energy storage device including, but not limited to an L-ion battery or a thin film battery. The pressure sensors 104 may also connect to the processor 114 using electronic conditioning (amp, offset, etc.). The processor 114 may also be connected to the wireless transmitter 118. The processor may be configured to connect to a computing system through the wireless transmitter. For example, the wireless transmitter may include a Bluetooth transceiver. In some embodiments, the housing may also function as a handle for the device.

### II. Computing System

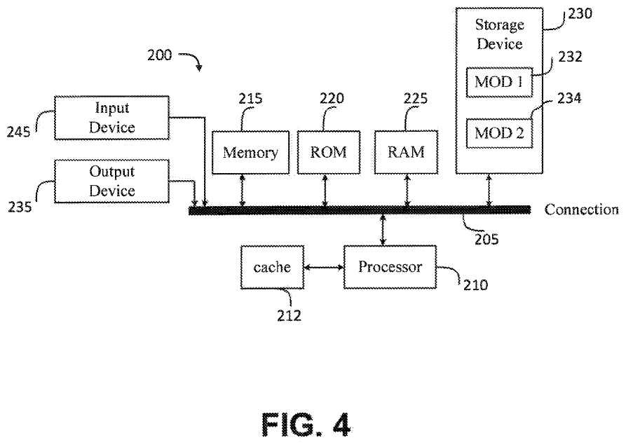

The pressure-sensing guidewire system may further include a computing system. FIG. 4 shows an example of computing system 200 in which the components of the system are in communication with each other using connection 205. Connection 205 can be a physical connection via a bus, or a direct connection into processor 210, such as in a chipset or system-on-chip architecture. Connection 205 can also be a virtual connection, networked connection, or logical connection.

In some examples, one or more of the described system components represents many such components each performing some or all of the function for which the component is described. In some examples, the components can be physical or virtual devices.

Example computing system 200 includes at least one processing unit (CPU or processor) 210 and connection 205 that couples various system components including system memory 215, read only memory (ROM) 220 or random access memory (RAM) 225 to processor 210. Computing system 200 can include a cache of high-speed memory 212 connected directly with, in close proximity to, or integrated as part of processor 210.

Processor 210 can include any general purpose processor and a hardware service or software service, such as an acquisition system 232 and data post-processing system 234 stored in storage device 230, configured to control processor 210 as well as a special-purpose processor where software instructions are incorporated into the actual processor design. Processor 210 may essentially be a completely self-contained computing system, containing multiple cores or processors, a bus, memory controller, cache, etc. A multi-core processor may be symmetric or asymmetric.

To enable user interaction, computing system 200 includes an input device 245, which can represent any number of input mechanisms, such as a touch-sensitive screen for gesture or graphical input, keyboard, mouse, or input from the sensing area 108. The input device 245 may be wired or wireless. Computing system 200 can also include output device 235, which can be one or more of a number of output mechanisms known to those of skill in the art. For example, the output device 235 may be a display. In some instances, multimodal systems can enable a user to provide multiple types of input/output to communicate with computing system 200. There is no restriction on operating on any particular hardware arrangement and therefore the basic features here may easily be substituted for improved hardware or firmware arrangements as they are developed.

Storage device 230 can be a non-volatile memory device and can be a hard disk or other types of computer readable media which can store data that are accessible by a computer, such as magnetic cassettes, flash memory cards, solid state memory devices, digital versatile disks, cartridges, battery backed random access memories (RAMs), read only memory (ROM), and/or some combination of these devices.

The storage device 230 can include software services, servers, services, etc., that when the code that defines such software is executed by the processor 210, it causes the system to perform a function. In some examples, a hardware service that performs a particular function can include the software component stored in a computer-readable medium in connection with the necessary hardware components, such as processor 210, connection 205, output device 235, etc., to carry out the function. In some examples, the storage device 230 includes an acquisition system 232 and a data post-processing system 234.

The acquisition system 232 may include instructions to cause the processor 210 to receive the measurements from the two or more pressure sensors 104. In some embodiments, the acquisition system 232 may be in communication with the wireless transmitter in the housing 110 of the pressure-sensing guidewire device 100. The acquisition system 232 may have an acquisition speed of at least 16 Hz. In an embodiment, the acquisition system may have an acquisition speed of at least 18 Hz. In an embodiment, the acquisition system may have an acquisition speed of at least 20 Hz.

The data post-processing system 234 may include instructions to cause the processor 210 to process the pressure measurements acquired from the acquisition system 232. For example, the data post-processing system 234 may denoise the measurements from one or more pressure sensors 104 located in the sensing area 108 using the measurements from one or more pressure sensors 104 in the intermediate portion 111. In various examples, the denoising may be by subtraction, advanced filtering, functional dependencies between the first and second sensor waveforms, and/or any denoising algorithm known in the art. The data post-processing system 234 may further generate a mapping of the coaptation force between the two tissues. In some embodiments, the data post-processing system may generate a 1D, 2D, or 3D map of the coaptation force. In other examples a “continuous, real time” dimension may be integrated with the 3D mapping to generate a 4D mapping of the coaptation.

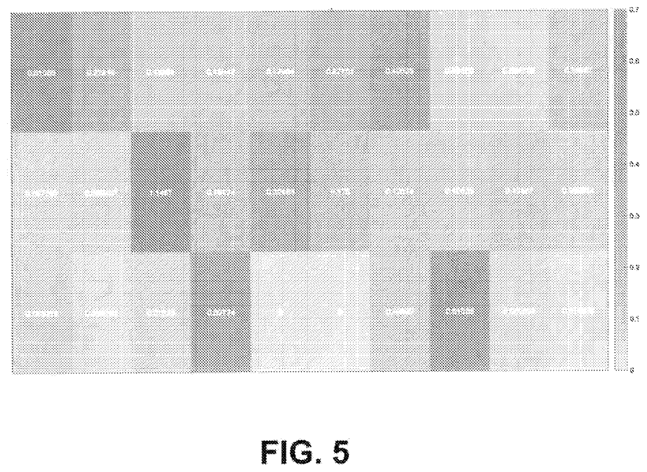

The denoised measurement and/or generated mapping of the coaptation force from the data post-processing system 234 may be output to the output device 235, such as a display. FIG. 5 is an example display of a 2D mapping generated from the acquisition system and the data post-processing system. The visual display may be used by the physician to easily identify where the pressure at a point or area on the contact surface may be problematic. For example, the mapping allows the physician to assess the MCF, allowing identification of abnormal coaptation or zones of coaptation. In some aspects, the physician may correct an aspect of the surgical repair based on the information in the visual display. In some embodiments, the visual display may be a table, a graph, a one-dimensional (1D) map, or a two-dimensional (2D) map of the pressures measured from the two or more pressure sensors. In various embodiments, the map may be color coded to indicate pressure values.

III. Methods of Use

Further provided herein are methods of using the pressure-sensing guidewire system 100, including methods of measuring a coaptation force.

The method of measuring a coaptation force of two tissues in a patient may include positioning a pressure-sensing guidewire device between the two tissues and measuring, with the two or more pressure sensors, a coaptation force between the two tissues.

Positioning the pressure-sensing guidewire device may include positioning a first pressure sensor between the two tissues and positioning a second pressure sensor outside the two tissues.

Measuring the coaptation force between the two tissues may include receiving a first pressure measurement from the first pressure sensor and a second pressure measurement from the second pressure sensor and denoising the first pressure measurement, using the second pressure measurement, to generate the measurement of the coaptation force of the two tissues. In various examples, the denoising may be by subtraction, advanced filtering, functional dependencies between the first and second sensor waveforms, and/or any denoising algorithm known in the art. The denoised first pressure measurement may then generate the measurement of the coaptation force of the two tissues. In some examples, more than one pressure sensor may be located between the two tissues, and each pressure sensor between the tissues may be denoised using the measurement of any pressure sensors not between the two tissues.

The method may further include inserting the pressure-sensing guidewire device during T-MVR. In an embodiment, the method may include inserting the pressure-sensing guidewire device endovascularly. In some examples, the pressure-sensing guidewire device may be inserted after the patient has had at least one of the two tissues repaired. The two tissues may be valvular tissues in the heart, such as leaflets of a mitral valve, leaflets of an aortic valve, leaflets of a pulmonary valve, or leaflets of a tricuspid valve. In some examples, the pressure-sensing guidewire device may be inserted between two leaflets of the mitral valve before, during, and after a mitral valve repair. In another example, at least one leaflet of the mitral valve may be repaired and the pressure-sensing guidewire device measures the coaptation force between the mitral valve leaflets after repair. In yet another example, the pressure-sensing guidewire device may be inserted between two leaflets of another valve (e.g., tricuspid, aortic valve, etc.). The pressure-sensing guidewire device may measure the pressures in vivo. The pressure-sensing guidewire device may measure the plurality of pressures in a beating heart. In some embodiments, the heart may be on cardiopulmonary bypass while measuring the coaptation force. In other embodiments, the heart may be filled with blood while measuring the coaptation force. In other embodiments, the pressure-sensing guidewire device measures the plurality of pressures ex vivo. MCF may serve as a prognostic parameter during preoperative or postoperative follow-up, helping cardiologists to define optimal time for invasive MVR, and identifying patient-tailored strategy.

The method may further include monitoring the position of the pressure-sensing guidewire device and the at least two pressure sensors. For example, monitoring the position of the device may include using fluoroscopy, ultrasound, a pressure waveform from the at least two pressure sensors, or a combination thereof. The radiopaque distal tip of the device may enhance the monitoring of the location of the device in the patient.

The method may further include generating a visual representation of the coaptation force. In an embodiment, the method may include generating a 1D or 2D map of the coaptation force between the two tissues from the measured pressures. In some embodiments, the method may further include comparing elements of the visual representation to determine the efficacy of the coaptation. The coaptation force may be compared to a predetermined value and the computing system may notify the physician if the repair to the tissue is insufficient.

The pressure-sensing guidewire device is sensitive enough to detect a wide range of pressures between the tissues. The method may further include measuring down to a minimum pressure of 2 gF. This sensitivity may allow for a more complete understanding of the coaptation force and any repairs to the tissues.

The pressure-sensing guidewire system may also capture the pressures, and therefore the coaptation force, in real time. The acquisition system of the computing system may have an acquisition speed of at least 16 Hz. In various embodiments, the pressure-sensing guidewire device measures the plurality of pressures at least as fast as the rate of the beating heart.

In various aspects, the patient may be in surgery when the pressure-sensing guidewire device is placed within the patient. For example, the surgery may be needed to repair a valve in the heart. In an embodiment, the pressure-sensing guidewire device may be placed through an incision in the patient's heart. The pressure-sensing guidewire device may be placed at the beginning of surgery to understand lesions in the tissue, during repair of a valve, or after repair of a valve.

The method may further include inserting the device endovascularly. In some embodiments, the heart may be under bypass as the pressure-sensing guidewire device is placed and as the plurality of pressures are measured. In an embodiment, the pressure-sensing guidewire device measures the plurality of pressures in a beating heart.

In other embodiments, the pressure-sensing guidewire device may be used for detecting closure or stenosis of tissues not related to a valve. The pressure-sensing guidewire device may be used to detect the adequacy of a closure or stenosis. The two tissues being measured for coaptation force may be a single tissue shaped to be able to surround the pressure-sensing guidewire device and act essentially as two tissues. For example, the tissues may be a sphincter (e.g. pyloric sphincter), a critical stenosis of a blood vessel, or a closure (e.g. a vascular closure).

### EXAMPLES

### Example 1: Recording Mitral Coaptation Force (MCF) with Existing Piezoresistive Sensors in Humans

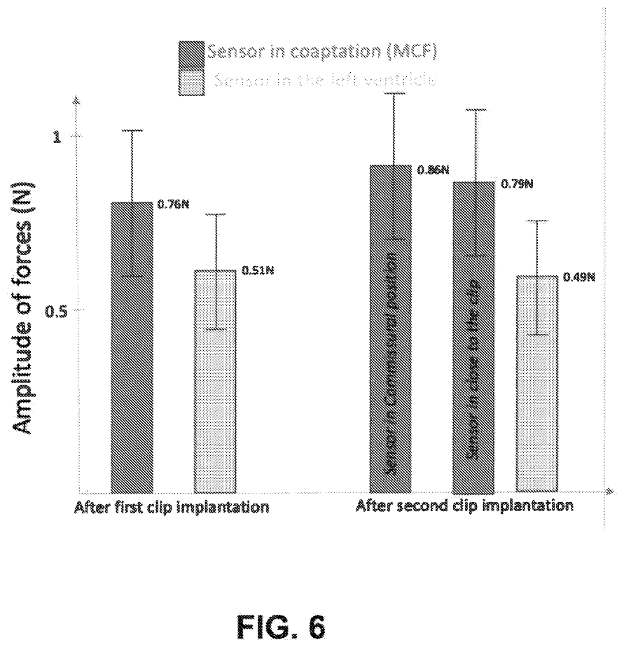

To determine if current technologies were able to record precise MCF data, a commercial FFR device (single sensing 0.1×0.1 mm surface area at the tip of a 0.0014″ guidewire) was used off-label to record clinical MCF. The sensor was successfully navigated to the LA and into the mitral valve. No device-induced regurgitation was seen on echocardiography while the sensor was within the valve. The results in FIG. 6 show mean force amplitudes were greater in the valve compared to the LV, and a mean increase in MCF following MitralClip™ implantation was recorded. A high degree of variability was observed, however, and no findings were found to be significantly different. This demonstrates that (1) guidewire sensors can be safely inserted within the mitral valve without device-induced regurgitation, and (2) existing technologies are fundamentally unable to capture MCF data of a precision required for surgeons to make informed decisions.

### Example 2: Endovascular Prototype and In Vivo Testing

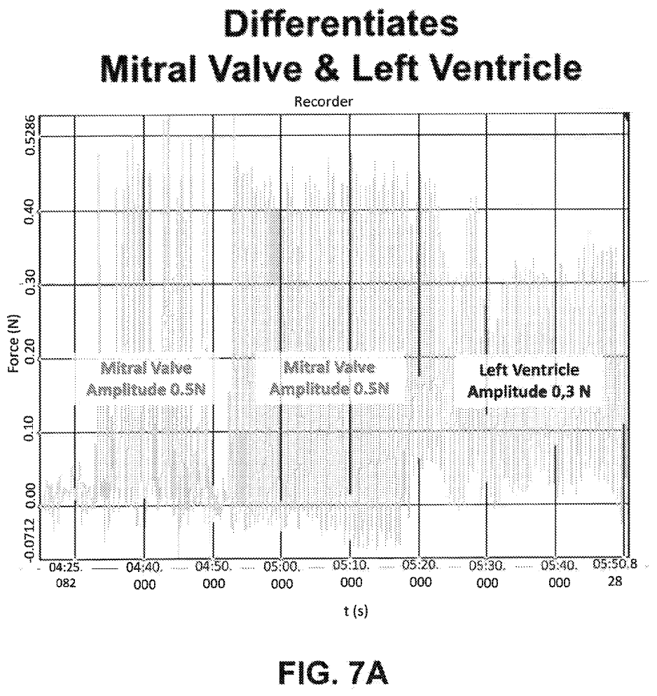

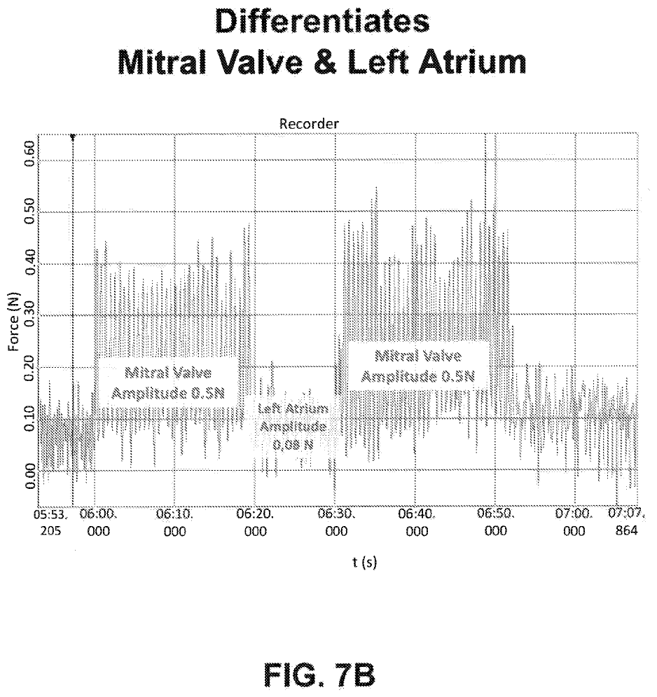

An early-stage endovascular pressure-sensing guidewire prototype was developed by transforming the previously described commercial piezoresistive FFR guidewire devices into a pressure-sensing guidewire with the addition of a coating of epoxy-glue. A proof-of-concept was obtained in a single in vivo experiment of an open-chest, beating-heart, healthy swine model. The sensor was inserted through a transapical puncture and placed within the mitral valve under fluoroscopic guidance. FIGS. 7A and 7B show the sensor was able to successfully detect a “MCF signal print,” with clear differentiation between valvular, atrial and ventricular signal. This prior work demonstrates the ability for a piezoelectric guidewire to be safely navigated to within the mitral valve, to obtain MCF measurements, and to differentiate MCF levels.

The particular variations disclosed above are illustrative only, as the variations may be modified and practiced in different but equivalent manners apparent to those skilled in the art having the benefit of the teachings herein. It is therefore evident that the particular variations disclosed above may be altered or modified, and all such variations are considered within the scope and spirit of the application. Accordingly, the protection sought herein is as set forth in the description. Although the present variations are shown above, they are not limited to just these variations, but are amenable to various changes and modifications without departing from the spirit thereof. Additionally, a number of well-known processes and elements have not been described in order to avoid unnecessarily obscuring the present invention. Accordingly, the above description should not be taken as limiting the scope of the invention.

Those skilled in the art will appreciate that the presently disclosed variations teach by way of example and not by limitation. Therefore, the matter contained in the above description or shown in the accompanying drawings should be interpreted as illustrative and not in a limiting sense. The following claims are intended to cover all generic and specific features described herein, as well as all statements of the scope of the present method and system, which, as a matter of language, might be said to fall therebetween.

What is claimed is:

1. A device for measuring a coaptation force of two tissues in a patient comprising:
a guidewire body comprising at least two cavities at a distal end of the guidewire body;
at least two pressure sensors, each pressure sensor configured to fit within a corresponding cavity on the guidewire body; and
a flexible coating covering the at least two pressure sensors;
wherein the at least two pressure sensors are configured to provide a measurement of the coaptation force of the two tissues.

2. The device of claim 1, wherein the flexible coating fills a gap in each cavity between the top of each pressure sensor and a surface of the guidewire body.

3. The device of claim 2, wherein the flexible coating is flush with the surface of the guidewire body.

4. The device of claim 2, wherein the flexible coating extends above the surface of the guidewire body.

5. The device of claim 2, further comprising a second coating configured to cover the flexible coating over the at least two pressure sensors and the surface of the guidewire body.

6. The device of claim 2, wherein each cavity has a depth of 0.10 mm to 0.13 mm.

7. The device of claim 6, wherein each pressure sensor has a height of 0.05 mm to 0.08 mm.

8. The device of claim 7, wherein the gap between the top of each pressure sensor and the surface of the guidewire body has a depth of 0.02 mm to 0.08 mm.

9. The device of claim 1, wherein the at least two pressure sensors comprises three pressure sensors.

10. The device of claim 1, wherein the at least two pressure sensors comprises four pressure sensors.

11. The device of claim 1, wherein at least one of the at least two pressure sensors is located along a sensing area at the distal end of the guidewire body, wherein at least one pressure sensor in the sensing area is configured to measure mechanical pressure between the two tissues.

12. The device of claim 11, wherein at least one of the at least two pressure sensors is located outside of the sensing area at the distal end of the guidewire body, wherein the pressure sensor outside the sensing area is configured to measure blood pressure outside the sensing area.

13. The device of claim 12, wherein a measurement from the at least one pressure sensor outside the sensing area is used to denoise a measurement from the at least one pressure sensor in the sensing area to generate the measurement of the coaptation force of the two tissues.

14. The device of claim 12, wherein the sensing area is 1 cm to 1.5 cm long.

15. The device of claim 12, wherein the at least one pressure sensor outside the sensing area is proximal to the sensing area along the guidewire body.

16. The device of claim 12, wherein the at least one pressure sensor outside the sensing area is 1 cm to 2 cm from the sensing area.

17. The device of claim 12, further comprising a radiopaque tip distal to the sensing area.

18. The device of claim 17, further comprising a radiopaque marker proximal to the sensing area.

19. The device of claim 1, wherein the at least two pressure sensors are piezoresistive force sensors.

20. The device of claim 1, wherein the at least two cavities each comprise a sensor housing.

21. The device of claim 1, further comprising a handle configured to hold a processor, a battery, and a wireless transmitter.

22. The device of claim 1, wherein the two tissues are valvular tissues within the patient's heart.

23. The device of claim 22, wherein the two tissues are leaflets of a mitral valve, an aortic valve, a pulmonary valve, or tricuspid valve.

24. A method of measuring a coaptation force of two tissues in a patient comprising:
positioning a pressure-sensing guidewire device between the two tissues, the pressure-sensing guidewire device comprising:
a guidewire body comprising at least two cavities at a distal end of the guidewire body;
at least two pressure sensors, each pressure sensor configured to fit within a corresponding cavity on the guidewire body; and
a flexible coating covering the at least two pressure sensors; and
measuring the coaptation force of the two tissues via the at least two pressure sensors.

25. The method of claim 24, further comprising inserting the pressure-sensing guidewire device endovascularly.

26. The method of claim 24, further comprising monitoring the position of the pressure-sensing guidewire device and the at least two pressure sensors.

27. The method of claim 26, wherein monitoring the position of the device comprises using fluoroscopy, ultrasound, a pressure waveform from the at least two pressure sensors, or a combination thereof.

28. The method of claim 24, further comprising positioning a first pressure sensor between the two tissues, wherein a second pressure sensor is not between the two tissues.

29. The method of claim 28, further comprising:
receiving a first pressure measurement from the first pressure sensor and a second pressure measurement from the second pressure sensor; and
denoising the first pressure measurement, using the second pressure measurement, to generate the measurement of the coaptation force of the two tissues.

30. The method of claim 24, wherein the two tissues are valvular tissues in the heart and the pressure-sensing guidewire device measures pressures in a beating heart.

31. The method of claim 30, wherein the two tissues are leaflets of a mitral valve, an aortic valve, a pulmonary valve, or a tricuspid valve.

32. The method of claim 24, further comprising generating a visual representation of the coaptation force between the two tissues from the measured pressures.

33. The method of claim 32, further comprising comparing elements of the visual representation to determine the efficacy of the coaptation.

34. The method of claim 24, wherein the two tissues comprise a sphincter.

35. The method of claim 24, wherein the two tissues comprise a stenosis of a blood vessel or a vascular closure.
